(19) United States
(12) Patent Application Publication
(10) Pub. No.: US 2026/0021210 A1
(43) Pub. Date: Jan. 22, 2026

(54) METHOD OF TOOTH MARKING

(71) Applicant: Rutgers, The State University of New Jersey, New Brunswick, NJ (US)

(72) Inventors: Michael CONTE, Brick, NJ (US); Nathalie LOTA, Franklin Lakes, NJ (US)

(21) Appl. No.: 19/273,672

(22) Filed: Jul. 18, 2025

Related U.S. Application Data
(60) Provisional application No. 63/672,740, filed on Jul. 18, 2024.

(51) Int. Cl. A61K 49/00

(52) U.S. Cl. CPC A61K 49/006

(57) ABSTRACT

The present invention relates to a method of marking a tooth or teeth for identification purposes by adhering a product to the tooth that is visible to the practitioner under UV (black) light. This disclosure provides a dental bonding agent containing a resin and a component that allows the material to glow under UV black light. In order to prevent wrong-site tooth extraction, the referring dentist, for example, can apply the inventive product to a particular tooth to mark and identify the tooth to be treated by another practitioner. This mark is not visible under normal circumstances (under normal lighting conditions) and therefore would not be noticeable, but identifies the particular tooth by glowing under black light.

### CROSS-REFERENCE TO RELATED APPLICATIONS

This application is a United States utility application, which claims the benefit of U.S. provisional application Ser. No. 63/672,740, filed 18 Jul. 2024. The entire contents of the aforementioned application(s) is/are hereby incorporated by reference as if fully set forth herein.

### BACKGROUND OF THE INVENTION

### 1. Field of the Invention The invention relates to the general field of dentistry, for example exodontia.

### 2. Background of the Invention

Exodontia is the dental procedure of tooth extraction. Dentists typically attempt to restore the health of a tooth to avoid removal, but in the case of teeth that are damaged, decayed, impacted, or infected, extraction of the tooth may be necessary. Tooth extractions are commonly performed by General Dentists, Oral and Maxillofacial Surgeons, and Periodontists, and therefore may not be performed by the dental practitioner diagnosing the dental problem and recommending the treatment or extraction.

When patients are referred to a dentist for tooth extraction, generally the teeth to be extracted are identified on a referral form. However, there are multiple tooth numbering systems between different dental specialties, which can lead to confusion as to the tooth that is planned for extraction. See Table 1, below.

TABLE 1
Notations for Tooth Identification of
the Upper Left first Molar Tooth.

| Notation System | Tooth |
|---|---|
| Long-hand | Upper left first molar |
| Palmer | /6 |
| FDI | 26 |
| Universal | 14 |
| Alphanumeric | UL6 |

Such differences can result in the wrong tooth being extracted. When the wrong tooth is extracted, patients bear the brunt of the error and must return for extraction of the correct tooth. Miscommunication in the referral and extraction or treatment of the wrong tooth can be attributed in large part to the differences in the tooth numbering systems in different dental specialties and the similarities between primary (baby) teeth and adult teeth.

Analysis of “never events” (defined as a ‘serious, largely preventable patient safety incident that should not occur if the available preventative measures have been implemented by healthcare providers.’) reported to the NHS in England show that there has been no reduction in frequency of wrong tooth extraction between 2015-2019. In 2016, such events were found to be the most common serious patient safety incident by the NHS, finding that 12% of clinicians had committed the error. Extraction of the wrong tooth therefore is a common event which has not been prevented by the safety measures currently in place.

Current practices in exodontia involve referral forms and records (both paper and electronic) to record the designated teeth for extraction. In 2017, a British Dental Journal article proposed several interventions to reduce wrong tooth extractions, including educational programs, patient-assisted identification, checklists, and tooth marking. See Cullingham et al. Tooth marking was encouraged by the American Dental Association, but JCAHO opined that a practical way to do so does not exist.

There is a long-felt need for methods to prevent misidentification of teeth, particularly in the context of exodontia.

### SUMMARY OF THE INVENTION

Thus, there exists a need in the art for a method of identifying a particular tooth for treatment or extraction. This invention allows the referring dentist to physically mark teeth requiring extraction or other treatment in a way that is not noticeable during day-to-day activities. The dentist performing the extraction, however, can clearly identify the tooth/teeth to extract or treat by simply using UV black light to illuminate the markings.

In particular embodiments, the present invention relates to a tooth-marking composition, comprising: a dental bonding agent and a melamine resin. Preferably, the dental bonding agent is self-etching. In certain embodiments, the dental bonding agent is a mixed two-part bonding agent comprising a part A and a part B.

In preferred embodiments, the ratio of dental bonding agent to melamine resin in the tooth marking composition is about 1.1 mg melamine resin powder to one drop each of part A and part B.

In additional embodiments, the invention includes a method of marking a tooth, comprising applying any of the compositions described herein to the tooth.

In certain embodiments, the invention includes a kit, comprising: a dental bonding agent and a melamine resin. Preferably, the dental bonding agent is a two-part bonding agent comprising a part A and a part B. Preferably, the dental bonding agent is self-etching.

In alternate kits according to the invention, the kit can contain a dental bonding agent such that the melamine resin is pre-mixed with one part of the bonding agent.

In some embodiments, the kit further comprises one or more of a mixing vessel, a written set of instructions, and an application brush.

### BRIEF DESCRIPTION OF THE DRAWINGS

Certain embodiments are illustrated by way of example, and not by way of limitation in the figures of the accompanying drawings.

FIG. 1 is a photograph of marked teeth under normal lighting.

FIG. 2 is a photograph of the same marked teeth under black light.

### DETAILED DESCRIPTION OF EMBODIMENTS OF THE INVENTION

### 1. Overview

The present invention relates to a method of marking a tooth or teeth for identification purposes. This method involves adhering a product to the tooth that is visible to the practitioner under UV (black) light.

### 2. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, clement or step or group of items, elements or steps. Furthermore, the indefinite article “a” or “an” is meant to indicate one or more of the item, clement or step modified by the article.

As used herein, the term “about” means plus or minus 20 percent of the recited value, so that, for example, “about 0.125” means 0.125±0.025, and “about 1.0” means 1.0±0.2. Notwithstanding that the numerical ranges and parameters setting forth the broad scope of the invention are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit.

Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

As used herein, the term “black (UV) light” refers UVA light with a wavelength of 320-400 nanometers. Certain fluorescent materials emit more visible light when they are “excited” by a black light.

As used herein, the term “dental bonding agent” refers to a thin layer of adhesive resin which is normally applied between the conditioned (etched) tooth structure and the resin matrix of composite filling materials in dentistry. Conditioned (etched) tooth structure can be achieved by applying an acidic substance to prepare the tooth for the application of the adhesive or by using a self-etching dental bonding agent.

As used herein, the term “self-etching,” in the context of dental adhesives, refers to a bonding compound that etches the tooth as well as bonding to it, without any further steps. This usually is accomplished by using a bonding composition with acid components. Dental self-etch systems have an acidic resin which etches and primes tooth structure without the need for etching or rinsing prior to the subsequent application of a tooth bonding agent.

### 3. Embodiments of the Invention

### A. Introduction

This disclosure involves a dental bonding agent containing a resin and a component that allows the material that glows under UV black light, as a solution for preventing wrong-site tooth extraction that is easy for dentists to adopt. The referring dentist, for example, can apply the inventive product to a particular tooth to mark and identify the tooth to be treated by another practitioner. This mark is not visible under normal circumstances (under normal lighting conditions) and therefore would not be noticeable, but identifies the particular tooth by glowing under black light. The treating dentist, therefore, can easily see which tooth is to be treated or extracted and be confident that the correct tooth is being treated.

The marking product comprises a dental bonding agent containing a resin that glows under black light which can be adhered to the specific tooth or teeth to be identified. Thus, the invention provides a composition and method for marking teeth.

### B. Bonding Agents

Any light-cured dental bonding agent known in the art which is compatible with the other components of the marking composition is contemplated for use with the invention. Preferably, a self-etching dental bonding agent such as Centrix Connexio® Self Etch Bond, 3M® Self Etch bonding system, and the like are used, but all light-cured dental bonding agents are compatible. Self-etching agents are more convenient. In dentistry, in order to bond a resin to tooth structure, the tooth is first etched via the application of an acid. When the acid is washed off the tooth, the conditioned tooth structure has an opaque appearance. This opaque appearance is the result of microscopic porosities chemically eroded into the tooth by the etch. Bonding agent (an unfilled resin) is then applied and cured with a light. This bonding agent is now attached to the tooth via the micro porosities. The layer of the bonding agent that is in contact with the air, (Oxygen inhibited layer) remains uncured and when the resin filling material is placed, the resin integrates with the oxygen inhibited layer of the bonding agent and both are cured with a light. Self-etch bonding systems eliminate the need to apply the acid etch and rinse step. The Connexio® and the 3M® products both come as two parts that are mixed, although the configurations are different.

Self-etch systems usually are acid, due to the presence of acidic monomers such as carboxylic or phosphoric acid groups, that etch the tooth structure to create a pattern that increases mechanical retention and to create chemical bonding by complex formation with the calcium ions present at the tooth. The acidity of the bonding agents varies considerably and can be significantly less acidic than of phosphoric acid.

These acidic adhesives simultaneously demineralize and penetrate into the surface of the tooth, but remain sealed. post-operative sensitivity is greatly reduced compared to total-etch adhesives. However, on the enamel surfaces, this reduced acidity can lead to a less pronounced etch pattern compared to phosphoric acid, especially on unprepared or uncut surfaces. Therefore, most self-etch adhesive systems require the incorporation of a separate phosphoric acid etch of these uncut enamel surfaces prior to applying the self-etch adhesives. Self-etch adhesives with a pH<2 are classified as “strong” self-etch adhesives, whereas those with a pH>2 are classified as “mild.” Mild self-etch adhesives are preferred over strong ones because they still provide for a strong bond to dentin, however their formulations are less hydrophilic and therefore less prone to undergo hydrolytic degradation, leading to improved shelf life and improved longevity of the restoration.

With the mild self-etch adhesive systems, including 3M™ Adper™ Easy One Self-Etch Adhesive, a “selective” enamel etch technique is often used and recommended on the enamel (cut and uncut) surfaces to maximize the adhesion to the more highly mineralized enamel. The dentin is not etched to take advantage of the self-etch properties on less mineralized dentin, leaving the surface sealed and offering protection from potential sensitivity.

### C. UV (Black) Light Visible Resin

Resins that are suitable for use with the invention include melamine resins or any resin that is UV fluorescent. For example, melamine copolymer resins can be used. Glo Germ™ is a UV light visible resin. This resin is a melamine resin according to the structure below:

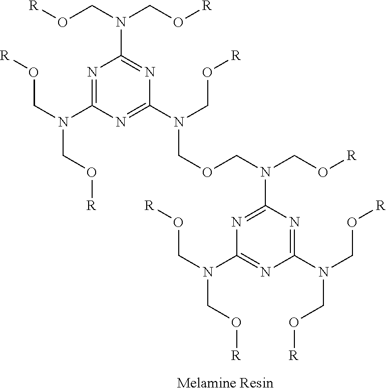

### D. Method of Preparation

The products of the invention comprise a mixture of bonding agent and UV (black) light resin. The products can contain bonding agent and resin (100% melamine) in a ratio of about 1 drop part A and 1 drop part B of the bonding agent to about 1.1 mg melamine resin powder. Preferably, the ratio can vary by about 10%, but the most preferred compositions use a ratio of bonding agent to resin of about 1 drop part A and 1 drop part B of the resin to about 0.5 mg to about 1.5 mg melamine resin.

The inventive compositions are made as follows: one drop each of part A and part B of Centrix Connexio Self Etch Bond (or other two-part dental bonding agent) were placed in a mixing palette or other vessel at room temperature. An application brush can be used for mixing or as convenient. 1.1 mg melamine resin was added to the bonding agent and mixed. The product then is ready for use and must be used within about 1-2 minutes or until the natural UV light begins to harden the product. Preferably, the product is use immediately after mixing.

### E. Method of Use

The inventive composition contains a dental bonding agent that allows a dentist or other practitioner to apply or paint the composition to the tooth or teeth and cure it in place. The composition adheres to the tooth and provides a mark visible only under black light to identify one or more teeth as desired. This mark will be easily seen by a second dental practitioner to avoid tooth misidentification.

After mixing, the composition is applied to clean dried enamel of a tooth to be marked, allowed to sit on the tooth (self-etch) for about 20 seconds and then light cured for about 20 seconds, or applied, rested, and light cured according to the manufacturer's instructions for the dental bonding agent. The material adheres to the tooth structure and illuminates very well under black light.

### F. Kits

Alternatively, compositions according to the invention can be sold as a kit. For example, the kit may include a dental bonding agent in one or two parts (i.e., one or two containers such as dropper bottles) and UV light visible resin (in a single larger container of bulk powder, or prepacked in individual pouches, capsules, or other small containers each containing about 1.1 mg melamine or sufficient to make the product for a single use), as well as, optionally, a mixing vessel, an application brush, and/or instructions for use, packaged together.

In an alternative embodiment, the product can be sold as two parts, including part A and part B of the dental bonding agent where the melamine resin is pre-mixed into either part A or part B of the dental bonding agent. In this case, the two parts are mixed together and are immediately ready for use.

### G. Conclusion

To prevent wrong-site tooth extraction, preferred embodiments of this invention provides a self-etching dental bonding agent, lightly filled with non-toxic melamine copolymer resin that glows under a black UV light. This composition and method allow referring dentists to mark the tooth/teeth planned for extraction or other treatment. Such markings will be unnoticed by the patient and others unless a UV black light is present, but the dentist performing the extraction or other treatment can compare the marked tooth/teeth with the documentation in the referral form to avoid mistaken identification of the correct tooth/teeth.

### 4. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1

### Tooth Marking Composition

To produce an embodiment of the tooth marking composition according to the invention, 1.1 mg of 100% melamine resin powder is added to and mixed into one drop each of part A and part B of a two-part self-etch bonding agent. The part A and part B then are mixed together immediately before use.

### Example 2

### Tooth Marking Product Use

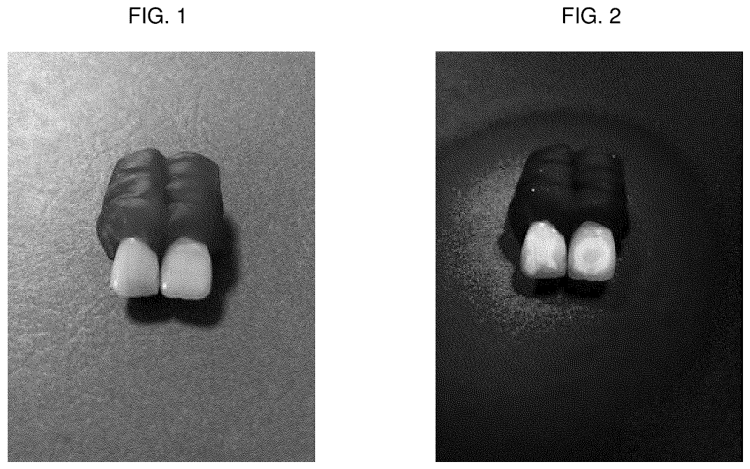

Immediately prior to use, the two parts of Centrix Connexio® Self Etch Bond dental bonding agent (which contain melamine resin powder), were mixed together and applied to the clean, dried enamel of the tooth to be marked. The composition was allowed to sit on the tooth undisturbed at ambient temperature for about 20 seconds and then light cured using a dental curing light at about 420 nm to about 540 nm wavelength for about 20 seconds (or according to manufacturer's instructions for the dental bonding agent used). The product adhered well to the tooth structure and illuminated very well under black light. See FIG. 1 and FIG. 2.

This test was repeated using 3M Self Etch bonding system with the same results.

### Example 3

### Effect of Melamine Powder on Flowable Composite

Glow Germ™ melamine powder was mixed into a flowable composite (a flowable dental composite of lower viscosity) and the mixture held in a UV light proof glass container for at least 5 months. The addition of the melamine powder did not cause the flowable composite to change its properties. Therefore, the bonding agent was not affected in terms of its ability to etch the tooth surface and in terms of the bond strength. This shows that a mixture of dental composite and melamine reason powder is shelf-stable for at least 5 months.

### REFERENCES

All publications listed below and throughout the specification are hereby incorporated by reference in their entirety. Nothing herein is to be construed as an admission that the invention is not entitled to antedate such disclosure by virtue of prior invention.
1. U.S. Pat. No. 8,609,741.
2. U.S. Pat. No. 8,106,110.
3. Yoo et al., Enhanced Mechanical Properties of Melamine-functionalized Boron Nitride Nanosheets Reinforced with Epoxy Nanocomposites for Dental Applications, J. Materials Sci. 57(38), 2022.
4. Gomes et al., Aging Protocols and their Effects on Bond Strength of Total-Etch and Self-Etch Adhesive Systems to Dentin, The Open Dentistry J. 14:408-415, 2020.
5. Cullingham et al., Patient Safety: reducing the risk of wrong tooth extraction. Br. Dent. J. 222:750-763, 2017.
6. Smith, Richard, Patient Safety Network, psnet.ahrq.gov/web-mm/mark-my-tooth, 2007.
7. Kaneshima et al., Using UV light for adhesive remnant removal after debonding of orthodontic accessories. Braz. Oral. Res. vol32.007, 2018.

1. A tooth-marking composition, comprising: a dental bonding agent and a melamine resin.

2. The tooth-marking composition of claim 1 wherein the dental bonding agent is self-etching.

3. The tooth-marking composition of claim 1 wherein the dental bonding agent is a mixed two-part bonding agent comprising a part A and a part B.

4. The tooth-marking composition of claim 1 wherein the ratio of dental bonding agent to melamine resin is about 1.1 mg melamine resin powder to one drop each of part A and part B.

5. A method of marking a tooth, comprising applying the composition of claim 1 to the tooth.

6. A kit, comprising: a dental bonding agent and a melamine resin.

7. The kit of claim 6 wherein the dental bonding agent is a two-part bonding agent comprising a part A and a part B.

8. The kit of claim 6 wherein the dental bonding agent is self-etching.

9. The kit of claim 7 wherein the melamine resin is pre-mixed with one part of the bonding agent.

10. The kit of claim 6 wherein the kit further comprises one or more of a mixing vessel, a written set of instructions, and an application brush.
